(19) United States
(12) Patent Application Publication
(10) Pub. No.: US 2025/0058056 A1
(43) Pub. Date: Feb. 20, 2025

(54) DEVICE FOR THE SUBCORTICAL INJECTION OF A SUBSTANCE

(71) Applicant: CETEV, Cambon-lès-Lavaur (FR)

(72) Inventors: Jan STEUPERAERT, Le FAGET (FR); Adeline RENIER, Pompertuzat (FR)

(21) Appl. No.: 18/722,421

(22) PCT Filed: Dec. 20, 2022
(86) PCT No.: PCT/EP2022/086890

§ 371 Date: Jun. 20, 2024

(30) Foreign Application Priority Data
Dec. 22, 2021 (FR) 2114202

(51) Int. Cl. A61M 5/34; A61M 5/32

(52) U.S. Cl. CPC A61M 5/343; A61M 5/3202; A61M 5/3287; A61M 2205/332

(57) ABSTRACT

The current invention relates to a device (1) for the subcortical injection of a substance into a plant comprising an injector head comprising (i) a needle holder (4) in which is held (ii) a distinctly straight needle (5) that defines the longitudinal axis (A) of the device—said needle (5) being pierced through its center along said axis (A) with a channel leading to at least one hole in the side of the needle (5) near it's end but not in the actual cutting end, which is intended to penetrate the plant—and (iii) a guide (3) in which there exists a cavity wherein the needle holder (4) and needle (5) can move along the longitudinal axis (A) of the device (1)—said cavity also having an opening so as to allow for the passage of the needle (5) so that it may be inserted into the bark of the plant; as well as an striking mechanism (10) capable of impacting the needle holder (4) with a power of less than 10 Joules so as to facilitate the insertion of the needle (5) under the bark of the plant.

This patent application claims the priority of the French patent application FR 2114202 filed on Dec. 22, 2022, and which is incorporated herein by reference.

### FIELD OF THE INVENTION

The present invention relates to the field of injection and, more specifically, to a device intended for the subcortical injection of a substance in a plant.

### PRIOR ART

The administration of a substance such an insecticide, antibiotic, fungicide, nutrient solution, or even a growth regulator to a plant is well-known in the field of plant treatments, especially in response to infestations by insects, bacterial or fungal infections, nutrient deficiencies, or growth problems.

Such an administration can be performed by injecting the substance directly into the plant, that is to say injected under the bark (i.e., subcortically) and as close as possible to the inner plant tissue, also called xylem, comprising fibers and a vascular system for the transportation of the sap. This sap vascular system then ensures the transportation of the substance to the targeted organs ranging from the trunk to the leaves and fruits.

Such an application via injection is particularly known from the international application WO/2020/208189 disclosing a device intended for the injection a substance under the bark of a plant comprising a body; a needle; a tank; actuators so as to enable the insertion of the needle and/or the injection of the substance; force sensors so as to measure the applied force of the device against the plant, the insertion force of the needle in the bark, and/or the pressurizing force of the volume of substance present in the tank; and a microcontroller so as to activate and modulate the actuators according to the forces measured by the sensors.

Now, it has been revealed that the device described in the international application WO/2020/208189 A1 may have experienced difficulties when injecting substances into trees with particularly hard xylem. In addition, handling said device has sometimes proven difficult for the user due to its weight and the applied force required to enable insertion.

### SUMMARY OF THE INVENTION

The inventors have now demonstrated that the use of a striker, as long as it provides an energy only superior to at least 1.0 Joule, enables the needle to be inserted without difficulty in all types of trees while also removing the need for considerable applied force—as could be the case when a device uses an actuator to insert the needle—and preventing additional damage to the tree's bark or cambium. Such an energy requirement is relatively low and rather unexpected given that commercial strikers provide around 10 Joules of energy for electromechanical strikers and up to 30 Joules for electropneumatic strikers.

Moreover, the inventors have developed a new device in which the injector head is integral with the body of the device and has, with it, a clearance of the order of one centimeter along the longitudinal axis of the device corresponding to the axis of insertion of the needle into the bark. Therefore, the device turns out to be particularly compact and easy to handle for the user, while also proving to be very effective and reliable in use.

Thus, the present invention relates to an injection device capable of allowing the injection of a substance into the hardest of plant barks while being more compact, more robust, and easier to use.

For this purpose, the present invention relates to an injection device, preferably intended for the subcortical injection of a substance into a plant, comprising:
A body.
An injector head comprising:
   A needle holder in which a substantially straight needle is held and defines the longitudinal axis of the device, said needle is pierced in its center by a channel opening through at least one hole on the side wall of the needle (i.e. the back), and therefore not at the end thereof intended to penetrate the plant;
   A guide which has a cavity in which the needle holder and the needle can move along the longitudinal axis of the device, which cavity has an opening so as to allow the passage of the needle and its insertion into the bark of the plant;
A tank containing the substance to be injected and connected to the needle's channel via the needle holder;
A first actuator capable of acting on the tank so as to pressurize the substance stored in the tank and to allow the injection of this substance through the needle to the plant via the channel,
a tool for supporting the device on the plant, which support tool is able to be positioned in the longitudinal axis of the device and is located close to the needle, preferably this support tool has an orifice allowing the needle to pass through,
a second actuator, able to act on the support tool so as to allow the extraction of the needle after its insertion into the plant.

The injection device according to the invention is characterized in that:
1) The support tool also acts as a protective sleeve for the end of the needle;
2) The second actuator is also capable of retracting the support tool as the needle is inserted into the plant; et
3) in that it also includes a striking mechanism capable of impacting the needle holder, with a power of less than 10 Joules, so as to allow the insertion of the needle into the plant.

Such minimal striking power is sufficient to insert the needle by percussion into the xylem of the hardest plants and, simultaneously, makes it possible to preserve the integrity of the plant.

According to a preferred embodiment, the needle holder and the needle are integral with the body of the device and have, with it, a clearance of between 2 and 15 mm along the longitudinal axis of the device, preferably between 3 and 10 mm or, more preferably, between 4 and 8 mm, typically around 5 mm.

The inventors have in fact established that a back-and-forth movement of the needle holder and needle of only a few millimeters along the longitudinal axis in association with a low impact force was sufficient to ensure a good insertion via percussion of said needle into the bark of the plant, while ensuring that the device is compact and light, appreciable for the user.

This connection of the needle/needle holder assembly with the body of the device is ensured by the guide. Now, this guide also constitutes a stop which makes it possible to limit the movement of the injection head relative to the body according to the clearance intervals defined previously.

The invention also relates to a method for treating a plant by subcortical injection of a substance, which method comprises the following steps:
Application, to the bark of a plant, of a device as described above,
activation of the latter, so as to allow the insertion of the needle, within the xylem of the plant, then to allow the subcortical injection of the substance, and
extraction of the needle from the xylem of the plant by activation of the second actuator of said device.

The invention finally concerns the use of a device as defined above with a view to allowing the injection of a substance under the bark of a plant.

### BRIEF DESCRIPTION OF THE DRAWINGS

FIG. 1 is a cross-sectional side view of an injection device according to the invention;

FIG. 2 is an enlarged image of the injector head in FIG. 1; and

FIG. 3 is a schematic representation of an embodiment of the needle holder

FIG. 4 is a schematic representation of another embodiment of the needle holder.

FIG. 5 is a cross-sectional side view of the injector head prior to starting the device.

FIG. 6 is a cross-sectional side view of the injector head at the beginning of the insertion step;

FIG. 7 is a cross-sectional side view of the injector head in the middle of the insertion step;

FIG. 8 is a cross-sectional side view of the injector head at the end of the insertion step.

### DETAILED DESCRIPTION OF THE INVENTION

The substance to be injected may be of several types and belong to the family of biocides (e.g. insecticides, antibiotics, antifungals, and/or antiparasitics), nutrients or even growth regulators or even plant defense stimulators. plants. Now, and due to the destination of the device according to the invention, the substance to be injected will rather be a phytosanitary substance.

As for the striking mechanism, it will preferably have a power greater than 1.0 Joules, preferably a power of between 1.2 and 8 Joules and, particularly preferably, between 1. 2 and 4 Joules.

Such a striking mechanism could be an electromechanical or electropneumatic system.

In addition, said striking mechanism could also be a direct or indirect percussion system.

As an example, the striking mechanism may be a rotary striker system. In such a system, however, the needle and the needle holder will not rotate in the longitudinal axis. Indeed, and as established by European patent EP 3332630 B1, the orientation of the incising edge of the needle relative to the direction of the fibers of the plant is important for the injection efficiency and especially to limit damage to the plant when inserting the needle.

Typically, this striking mechanism is dependent on an activation means, such as a trigger, operable by the user. This means of activation allows insertion to begin and it can also provide dead man type safety. In this case, and as long as it is not permanently activated, it will cause the device to shut down.

Concerning the support tool, it is advantageously associated with a guiding means in the longitudinal axis of the device. This guiding means ensures the maintenance and centering of the support tool along the longitudinal axis of the device.

Preferably, the support tool comprises a support tip capable of coming into contact with the bark of the plant. This support tip can take a flat shape when the bark is smooth (e.g. plane tree) or a three-dimensional shape, notably conical, when the bark is cracked (e.g. pine). Such a support tip can be made of any suitable material and in particular of plastic, in particular a polyamide such as nylon. In fact, nylon is a resistant material so that it will not wear or wear only slightly on contact by friction against the bark of the plant.

According to a preferred embodiment, the injection device further comprises a first force sensor capable of measuring the applied force of the support tool against the bark of the plant and at least one microcontroller which enables:
1) as soon as a minimum effort, corresponding to the activation value, is detected,

the activation of the second actuator so as to retract the support tool and allow the insertion of the needle into the bark of the plant. Advantageously, this activation value is at least 2 daN. Preferably, the activation of the second actuator makes it possible to retract the support tool at a speed of at least 10 mm/second, typically of the order of 20 mm/s.
2) as soon as the support force is less than a threshold value called the strike value,

the activation of the strike mechanism. Such a reduction in the support force results from the activation of the second actuator which would not be associated simultaneously with the insertion of the needle into the tree. In fact, this shift then leads to a distance of the support tool from the surface of the bark, which distance increases the risk that the needle will twist. Advantageously, this impact value is less than 250 N, preferably it is 150 N. Advantageously, the microcontroller reduces the speed of the second actuator so as to reduce the retraction speed of the support tool. Typically, this reduction in the retraction speed of the support tool is at least a factor of 2, preferably a factor of 4.

As for the striking mechanism itself, it will preferably have a power of between 1.2 and 4 Joules and, more preferably, between 1.2 and 2 Joules.

Advantageously, the microcontroller allows:
3) as soon as the support force becomes less than a limit value, deactivation of the second actuator. Such deactivation helps limit the risk of damage to the needle. Preferably, this limit value is less than 50 N. Advantageously, the microcontroller increases the power of the striking mechanism. Typically, this increase in the power of the striking mechanism is greater than or equal to 50%, preferably greater than or equal to 100%.

As for the striking mechanism, it will preferably have a power of between 2 and 8 Joules and, particularly preferably, between 2 and 4 Joules.

Advantageously again, the microcontroller allows:
4) as soon as the pressing force is lower than the limit value for more than 5 seconds, preferably for more than 2 seconds, deactivation of the striking mechanism.

The couple formed by this first force sensor and the microcontroller constitute essential elements for the optimal insertion of the needle into the xylem of the plant and therefore the operation of the device.

According to a preferred embodiment, the injection device further comprises a first position sensor, associated with the second actuator, capable of measuring the depth of insertion of the needle into the bark of the plant.

Advantageously, the microcontroller, in connection with the information transmitted by this first position sensor, allows:
1) once the target depth is reached, for the plant into which the needle is inserted, a deactivation of the second actuator and the striking mechanism. Advantageously, the microcontroller then allows the activation of the first actuator, capable of acting on the tank so as to pressurize the substance stored therein, and the injection of the substance through the needle up to the plant.

By way of example, such a target depth is 45 mm for a large tree such as the chestnut tree, the chestnut tree or the pine, 38 mm for a fruit tree such as the apple tree, or even 25 mm for a thin and hard tree such as the Cherry tree.

Advantageously, the microcontroller allows, following deactivation of the striking mechanism due to an applied force lower than the limit value for more than 5 seconds, preferably for more than 2 seconds:
2) as soon as a limiting depth is reached, for the plant in which the needle is inserted, activation of the first actuator, capable of acting on the tank so as to put the substance stored in it under pressure, thereby initiating the injection of the substance through the needle and into the plant.
3) as soon as a limiting depth is not reached, for the plant into which the needle is inserted, to inform the user that the insertion sequence has failed. Such information can be provided by means of a light and/or sound indicator.

Indeed, below a limiting depth, the injection of substance would risk damaging the cambial base due to the detachment of the bark induced by the injection pressure of the substance. For example, such a depth limit is 33 mm for a large tree such as the chestnut tree, chestnut tree or pine, 29 mm for a fruit tree such as the apple tree, or even 18 mm for a thin and hard tree such as the cherry tree.

he microcontroller can then activate the second actuator to extract the needle from the bark of the tree, so as to allow for the initiation of a new insertion/injection sequence at another location in the bark.

Regarding the needle itself, it will have a diameter of between 1 and 3 mm, preferably between 1.5 and 2 mm. Indeed, a small diameter promotes healing of the plant following the lesion created by the passage of the needle in the bark of the plant during the injection.

Regarding the length of said needle, it will be between 2 and 20 cm, preferably between 2 and 10 cm and, even more preferably, between 3 and 7 cm. As for its shape, it may have a cylindrical shape.

As for the at least one hole present on the side wall of the needle and therefore not at the end thereof intended to penetrate the plant, this can be positioned less than one centimeter from this end, see less than 5 millimeters from it, in particular less than 4 see less than 3 mm.

Now, the needle may include several distal holes which form as many outlets from the channel and which are capable of allowing the dispersion of the substance in the plant.

The needle is ideally held in position, in particular thanks to the support tool described below, and resists well when it is inserted by percussion into the bark of the plant.

To facilitate the placement of the needle in the needle holder and its replacement, the needle holder incorporates a frame. Indistinctly, this frame can either be an integral part of the needle holder (i.e., form only one part with it), or consist of an independent part which is fixed to the needle holder. In the latter case, the needle frame and the needle holder are associated with each other in a watertight manner (if necessary with the addition of a seal) and by a reversible fixing (e.g. screwing, socket or any form of quick release).

In the case of a frame forming an integral part of the needle holder, the needle is associated with the needle holder in a reversible manner (e.g. locking screw, circlip, screwing, etc.). In the case now where the frame can be separated from the needle holder, the needle can be associated with the frame in a reversible manner or not (e.g. bonding, welding, soldering, etc.).

The needle and the frame are combined over a length which could be between 5 mm and 35 mm, in particular between 15 mm and 25 mm, more particularly over a length of 20 mm.

In connection now with the guide which acts as a stop, this is perhaps associated with a return means which typically takes the form of a spring. This return means allows, after the needle holder has advanced along the longitudinal axis of the device after being struck by the striking mechanism, to bring it back towards the striking mechanism for a new percussion. Now, the inventors have shown that such a return means is not necessary and that the applied force against the plant when inserting the needle is sufficient to bring the needle holder back quickly against the striking mechanism.

Advantageously, this guide which acts as a stop is removable.

Preferably, this guide is pivotally mounted, relative to the body, around a transverse axis perpendicular to the longitudinal axis of the device. In this way, the user can replace a needle and/or the needle holder more simply and quickly.

Preferably, the pivoting of the guide also allows access to the tank of substance to be injected. It is then possible to refill this tank or to change it in the case where it is advantageously mounted mobile.

Concerning the tank, it is connected to the needle holder, and therefore to the needle and its channel, via a conduit which advantageously takes the form of a hose.

The tank preferably takes the form of a mobile container, in particular a syringe.

Regarding the first actuator capable of acting on the tank so as to pressurize the substance stored therein and to enable the injection of said substance through the needle via the channel and into the plant, it will ideally take the form of a jack.

According to a preferred embodiment, the injection device further comprises a second force sensor capable of measuring the pressurization force of the substance stored in the tank, and at least one microcontroller which allows:
1) to modulate the compression of the volume of substance present in the tank by the first actuator, and therefore the injection of this substance into the plant, as a function of the force measured by the second force sensor.
2) as soon as the pressurization force is too great, to inactivate the first actuator so as to interrupt the injection. Such excess pressure force corresponds to an effort greater than or equal to 100 daN, preferably greater than or equal to 200 daN. Such excess pressure may be due, for example, to a too hard area in the plant or to a blockage of the needle hole. There is then a risk of damaging the shaft by separation of the cambial base, which would cause irreversible damage over a large area and above all unacceptable in the context of a treatment, and possibly the tank, or even the device.
3) since the pressurization force is too low, to inactivate the first actuator so as to interrupt the injection. Such a pressure force that is too low corresponds to an effort less than or equal to 5 daN. Such a too weak pressurization effort may result, for example, from internal damage to the plant. There is then a risk of carrying out an ineffective injection and, above all, of unnecessarily dispersing phytosanitary products into the external environment.

In these last two cases of an interruption of the injection sequence, the microcontroller can also inform the user of this interruption. Such information can be provided by means of a light and/or sound indicator. The microcontroller can then activate the second actuator to extract the needle from the xylem of the tree, in order to initiate a new insertion/injection sequence at another location in the bark.

The device according to the invention can also include a second position sensor which is associated with the first actuator and which makes it possible to inform the user, and potentially the device, about the volume of substance introduced into the plant. On the basis of the information provided by this second position sensor, the microcontroller is able to interrupt the injection phase, once the desired volume of substance has been injected into the plant, or to record the volume of substance actually introduced in the case where the injection was interrupted for one of the aforementioned reasons.

Depending on the plant to be treated, which is specified by the user for example, it is thus possible to modulate the volume of substance injected.

The device is powered by a power source which takes the form of a connection to the sector or a battery.

The device may optionally include a memory containing the operating parameters of the device and the parameters to be used depending on the plant to be injected.

Ideally, the device according to the invention comprises an operating indicator, such as an indicator light (LED).

Advantageously, the device according to the invention comprises gripping means, such as handles to make it easier for the user to hold the device.

The device according to the invention may further comprise carrying means, such as straps to relieve the user of the weight of the device.

The device advantageously includes a visual interface, such as a control screen, possibly a touchscreen, through which the user can select the control parameters (strike power, rotation speed, force measurements, actuator motor intensity, etc.). The visual interface also alerts the user of battery charge, volume of substance in the tank, etc. The visual interface also warns the user of any operating anomaly of the device (e.g. excessive effort detected, leak in the hose, etc.).

The device also includes means of protecting the exterior body, such as protective pads protecting the device in the event of a fall.

The device includes usual USB type connectors and others for data transfer. Said transfer can also be carried out via a wireless connection such as WIFI or BLUETOOTH.

With regard to the method of treating a plant by injection using the device described above, the insertion step is preferably carried out with the power of the striking mechanism being greater than 1.0 Joule, typically between 1.2 and 8 Joules and, more preferably, between 1.2 and 4 Joules.

Finally, concerning the use of a device as described above in order to allowing the subcortical injection of a substance, the power of the striking mechanism is preferably greater than 1.0 Joule, typically between 1.2 and 8 Joules and, more preferably, between 1.2 and 4 Joules.

The invention may be better understood upon reading the following description, given solely by way of non-limiting example and made with reference to the appended figures, in which:

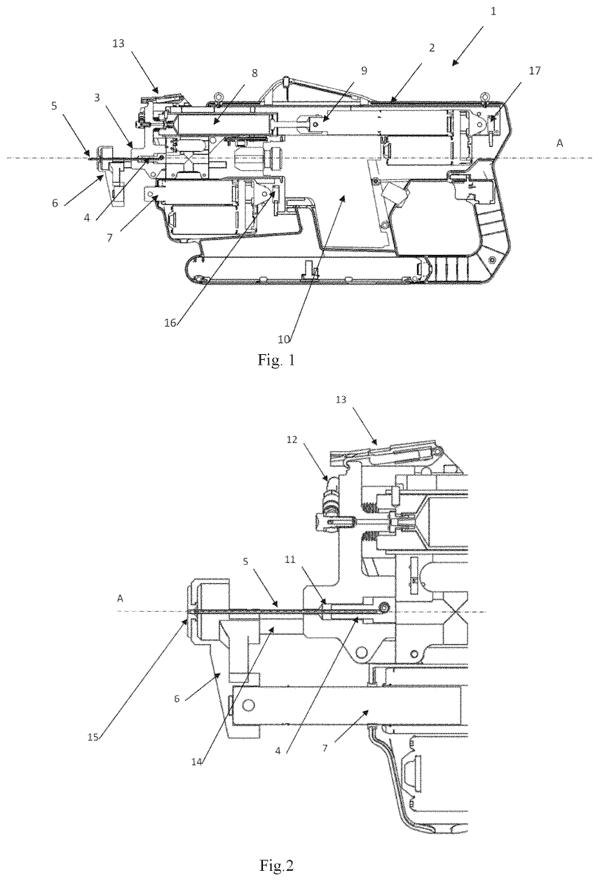

In FIG. 1, the injection device 1 is held horizontally and points towards its target. The front of the injection device 1 corresponds to the injector head, the rear of the device allows the user to hold it, in particular to apply a supporting force on the bark of a plant before the injection.

In reference to FIG. 1, the injection device 1 comprises a body 2, also called a chassis or frame, which corresponds to a machined part that serves as a support for the various constituent parts of said device. Said body can be perforated in different places so as to be as light as possible.

At the front end of the injection device 1, a locking/unlocking system 13, for example a latch equipped with a hook, attaches to a receptacle corresponding to the stop 3 of the injector head, and this in order to lock or secure the installation of the needle holder 4/needle 5 assembly.

This stop 3 is in the form of a protruding cylinder and in which a cavity is machined which is a counter form of the needle holder 4. At the center of said cylinder is also machined a hole that connects to said cavity and constitutes an opening through which a needle 5 can pass.

Needle 5 defines a longitudinal axis A of the injection device 1.

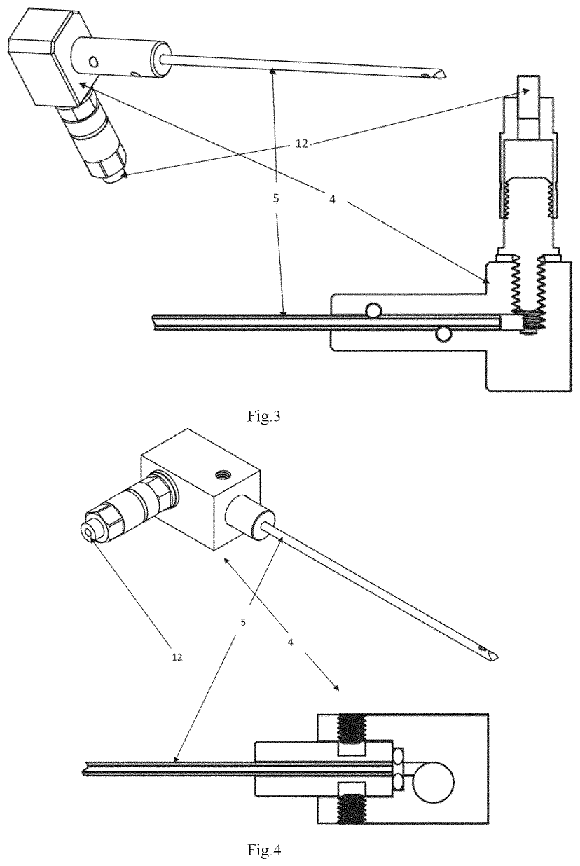

In the counterform, and when the stop 3 is fixed or secured on the body 2 of the injection device 1, a possible structure of needle holder 4 is shown in FIG. 3. In this needle holder, the needle frame is an integral part of the needle holder and is in the form of a hollow cylinder, for example with a length of 20 mm and a diameter of 5 mm, in which a drilling is machined which constitutes the housing of the needle 5. The channel of the needle 5, once the needle 5 is placed in the frame, opens into another channel machined within the needle holder 4, which other channel is connected, via the hose 12, to the tank 8 of the device 1. This other channel further comprises a means for immobilizing the needle 5, which takes the form of a screw which abuts on the needle 5, which screw is pierced in its center so as to allow the substance of the hose 12 to pass towards the needle channel 5.

Another possible structure of needle holder 4 is shown in FIG. 4. In this other needle holder 4, the mounting of the needle 5 always corresponds to a hollow cylinder in which a drilling is machined in which the needle 5 is immobilized (e.g. by gluing). Now, this frame is housed, with an O-ring interface for watertightness, in a complementary shape in the body of the needle holder 4 so as to allow the assembly of the needle holder 4. The locking of the frame in the needle holder 5 is secured by a grub screw. The needle channel 5, once the needle 5/needle holder mount assembly is in place, opens into another channel machined within the needle holder 4, which other channel is connected, via the hose 12, to tank 8 of device 1.

The guide 3 of the injector head is also pivotally mounted relative to the body 2 of the injector device 1 around a transverse axis B perpendicular to the longitudinal axis A. Thus, when the user unlocks the locking system 13 of the guide 3, the guide 3 tilts by gravity around the transverse axis B. A detection contact of the guide 3 on the body 2 is broken, thus securing the injection device 1.

The user is then able to fill the tank 8, located at the top of the device 1 and along the longitudinal axis A, with a substance or, more simply, to change it, when this tank 8 is removable.

Once the guide 3 is locked back in place on the device 1, the fixing system being locked, the connection between the hose 12 and the tank 8, is made through the upper part of the guide 3 by a connection machined within of the guide 3 which allows a watertight connection between the outlet of the tank 8 and the inlet of the hose 12.

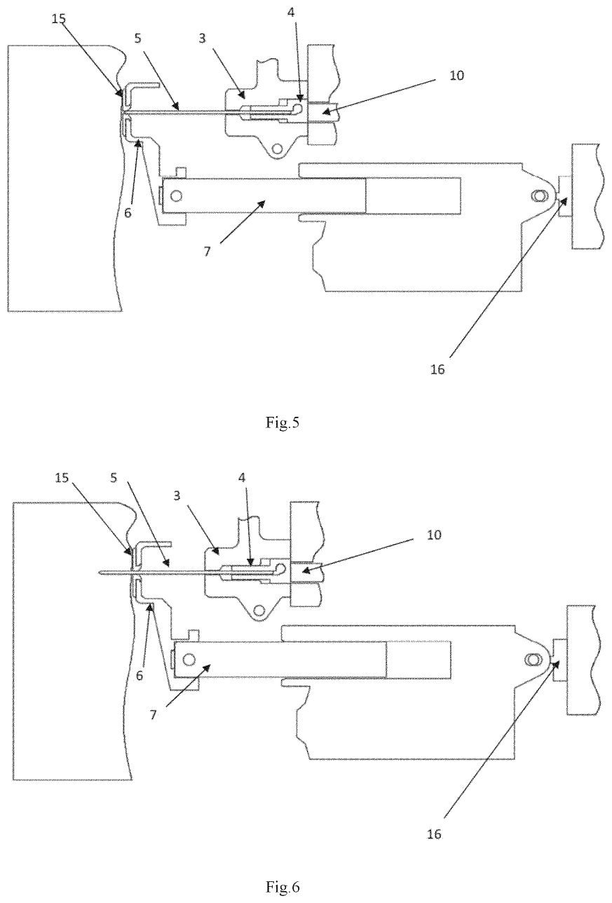

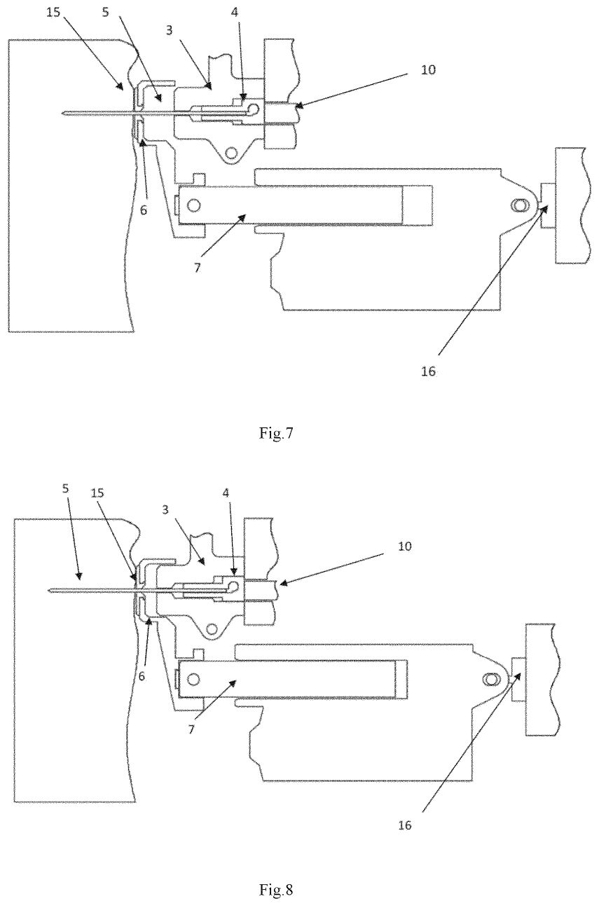

As illustrated in FIGS. 5 to 8, the user first applies the support tool 6, and more specifically a support jack 15, positioned in the longitudinal axis A, on the bark of the plant. This support jack 15 has a shape adapted to the type of bark, particularly flat for smooth bark. The support jack 15 can also be machined with a hole for the passage of the needle 5. The support tool 6 is associated with a force sensor 16 which measures the support force of the support tool 6 against the bark of the plant.

As soon as a minimum of force, corresponding to the activation value, is detected by the first force sensor 16, the microcontroller activates the second actuator 7 so as to retract the support tool 6 (at a speed of 20 mm/s) and enable the insertion of the needle 5 into the bark of the plant.

Needle 5 then gradually sinks into the bark of the plant. Now, if the needle 5 sinks into the plant less quickly than the second actuator 7 retracts the support tool 6, this results in a decrease in the applied force detected by the first force sensor 16.

When the applied force detected is below a threshold value, called the impact value, of typically 150 N the microcontroller activates the striking mechanism 10 (detail of said mechanism not shown in FIG. 1), which impacts the needle holder 4 along the longitudinal axis A of the device 1, giving it a back-and-forth movement within the guide 3, which allows the insertion of the needle 5 into the bark of the plant.

During this back-and-forth movement, the applied force brings the needle holder 4 back towards the striking mechanism 10. This striking of the striking mechanism 10 is generated by a mechanical or pneumatic transmission. A striker is propelled by a piston whose back-and-forth movement is kinematically controlled or by compressed air.

In this striking mechanism 10, the striker therefore strikes a chisel, in free translation in its housing, which then strikes the needle holder 4 in the longitudinal axis A. This mechanism is an indirect percussion system.

Alternatively, the chisel is no longer in free translation in its housing but in contact with the needle holder 4. The striking energy generated by the striker in contact with the chisel is then transferred optimally to the needle holder 4. It is a more efficient indirect percussion system.

When the striking mechanism 10 is a direct percussion system, the striker directly impacts the needle holder 4 in the longitudinal axis A. The striking energy generated by the striker in contact with the needle holder 4 is more important.

The direct percussion system is therefore more efficient than the indirect percussion system.

In these percussion systems, the striking energy generated by the striker in contact with the chisel or the needle holder 4 is transferred to the bark of the plant during the insertion by percussion of the needle 5 in this one.

During the various tests carried out, a power of the striking mechanism 10 of between 1.2 and 2 Joules was generally used. Simultaneously, the microcontroller reduces the speed of the second actuator 7 (typically 5 mm/s) so as to reduce the retraction speed of the support tool 6 and limit the risks of twisting the needle 5.

If the applied force becomes greater than the impact value again, the microcontroller deactivates the striking mechanism 10 and returns the retraction speed of the tool 6 to its initial speed via the second actuator 7. Now, and in certain situations, the applied force, after having been lower than the impact value, continued to decrease, and this despite the activation of the striking mechanism 10. From then on, a limit value was defined (generally of 50 N), below which the microcontroller deactivates the second actuator 7 and, simultaneously, increases the power of the striking mechanism 10 (generally between 2 and 4 Joules).

This sequence makes it possible to improve the insertion of the needle 5 and is stopped as soon as the applied force returns above this limit value.

Once the needle 5 has been inserted to a target depth in the xylem of the plant, which target depth is identified by a first position sensor, the microcontroller can initiate the injection of the substance.

To do this, the actuator 9 acts on the tank 8, under the control of the force sensor 17 thereto to measure the force of pressurization of the substance to be injected into the xylem of the plant. The substance then passes from tank 8, enters hose 12 to needle 5 via needle holder 4, before being injected into the plant.

Once the necessary volume of substance has been injected, as determined by the second position sensor associated with the first actuator 9, the actuator 7 acts on the support tool 6 to advance the support tool 6 against the bark of the plant in the longitudinal axis A until the needle 5 completely emerges from the bark of the plant.

To this end, the support tool 6 comprises, in the lower part and along the longitudinal axis A, a housing intended to accommodate the rod of the actuator 7. Preferably, the housing of the part lower part of the support tool 6 is magnetized so that the rod of the actuator 7 is in non-permanent magnetic connection with the support tool 6. Such a configuration makes it possible to disengage easily and without tools, the rod of the actuator 7 and the support tool 6.

During this extraction step, a guide means 14, for example slides, accompanies the support tool 6 along the longitudinal axis A of the device 1. Thus, two slides each arranged in on either side of the lower part of the injection head, along and parallel to the longitudinal axis A, slide in rails fixed inside the body 2 of the injection device 1 thanks to pebbles or balls.

During this same extraction step, one can imagine means of protection, for example protective bellows, aimed at protecting the rod of the actuator 7.

Once the extraction is completed, the support jack 15 can be removable in the support tool 6 so that the user can change the support jack 15 without detaching the tool from support 6 of the slides.

1. An injection device (1), preferably intended for the subcortical injection of a substance into a plant, comprising:
A body (2),
An injector head comprising:
A needle holder (4) in which a substantially straight needle (5) is held and defines the longitudinal axis (A) of the device, said needle (5) is pierced in its center by a channel opening through at least one hole on the side wall of the needle (5), and therefore not at the end thereof intended to penetrate the plant;
A guide (3) which has a cavity in which the needle holder (4) and the needle (5) can move along the longitudinal axis (A) of the device (1), which cavity has an opening so as to allow the passage of the needle (5) and its insertion into the bark of the plant;
A first actuator (9) capable of acting on a tank (8) so as to pressurize the substance stored in the tank (8) and to allow the injection of this substance through the needle (5) to the plant via the channel,
a support tool (6) from the device (1) on the plant, which support tool (6) is able to be positioned in the longitudinal axis of the device and is located close to the needle (5), preferably this support tool has an orifice allowing the needle (5) to pass through,
a second actuator (7), able to act on the support tool (6) so as to allow the extraction of the needle (5) after its insertion into the plant
Characterized in that:
The support tool (6) also acts as a protective sleeve for the end of the needle (5);
The second actuator (7) is also capable of retracting the support tool (6) as the needle (5) is inserted into the plant; and
The injection device (1) also includes a striking mechanism (10) capable of striking the needle holder (4), with a power of less than 10 Joules, preferably greater than 1.0 Joule, so as to allow the insertion of the needle (5) into the plant.

2. The injection device (1) according to claim 1, characterized in that the striking mechanism (10) is an electromechanical or electropneumatic system.

3. The injection device (1) according to claim 1, characterized in that the needle holder (4) and the needle (5) are integral with the body (2) of the device and have, with it, a clearance of between 2 and 15 mm along the longitudinal axis (A) of the device (1).

4. The injection device (1) according to claim 1, characterized in that the support tool (6) comprises a support jack (15) capable of coming into contact with the bark of the plant.

5. The injection device (1) according to claim 1, characterized in that it further comprises a first force sensor (16) capable of measuring the applied force of the support tool (6) against the bark of the plant and at least one microcontroller which enables:
i) as soon as a minimum effort, corresponding to the activation value, is detected, the activation of the second actuator (7) so as to retract the support tool (6) and allow the insertion of the needle (5) into the bark of the plant;
ii) as soon as the applied force is less than a threshold value called the strike value, the activation of the strike mechanism (10);
iii) as soon as the applied force becomes less than a limit value, deactivation of the second actuator (7).

6. The injection device (1) according to claim 1, characterized in that it further comprises a first position sensor, associated with the second actuator (7), capable of measuring the depth of insertion of the needle (5) into the bark of the plant, and at least one microcontroller allowing, once the target depth is reached for the plant into which the needle (5) is inserted, a deactivation of the second actuator (7) and the striking mechanism (10) and, preferably the activation of the first actuator (9) capable of acting on the tank (8) so as to pressurize the substance stored therein, and the injection of the substance through the needle (5) up to the plant.

7. The injection device (1) according to claim 1, characterized in that the striking mechanism (10) has a power of between 1.2 and 8 Joules, preferably between 1.2 and 4 Joules.

8. The injection apparatus (1) according to claim 1, characterized in that the guide (3) is pivotally mounted, relative to the body (2), around a transverse axis (B) perpendicular to the longitudinal axis (A) of the device.

9. The injection device (1) according to claim 1, characterized in that the needle holder comprises a frame can either be an integral part of the needle holder (4) or consist of an independent part which is fixed to the needle holder (4).

10. A method for treating a plant by subcortical injection of a substance; said method comprising the following steps:
application of a device (1), as defined in claim 1, to the bark of the plant,
activation of said device (1) so as to allow the insertion of the needle (5) under the bark of the plant and the subsequent subcortical injection of the substance, and
extraction of the needle (5) from the plant by activation of the second actuator (7) of said device (1).

11. The use of a device (1) as defined in claim 1 for allowing the injection of a substance under the bark of a plant.
